(19) United States
(12) Patent Application Publication
(10) Pub. No.: US 2024/0071584 A1
(43) Pub. Date: Feb. 29, 2024

(54) MEDICAL INFORMATION SYSTEM AND MEDICAL IMAGE DIAGNOSTIC DEVICE

(71) Applicant: CANON MEDICAL SYSTEMS CORPORATION, Otawara-shi (JP)

(72) Inventors: Shinya OZAWA, Saitame (JP); Kensuke SHINODA, Otawara (JP); Junichiro ARAOKA, Nasushiobara (JP); Tsutomu IGARASHI, Otawara (JP); Masato SHIBUYA, Otawara (JP)

(73) Assignee: CANON MEDICAL SYSTEMS CORPORATION, Otawara-shi (JP)

(21) Appl. No.: 18/457,859

(22) Filed: Aug. 29, 2023

(30) Foreign Application Priority Data
Aug. 30, 2022 (JP) 2022-136553

(51) Int. Cl. G16H 10/60; G16H 30/20

(52) U.S. Cl. CPC G16H 10/60; G16H 30/20

(57) ABSTRACT

A medical information system includes a processor on a side of a medical image diagnostic device. The processor acquires supplementary information as structured information associated with image data acquired using the medical image diagnostic device, the supplementary information being new information on a subject to be imaged. The processor notifies at least one management server that manages patient information of the supplementary information via a network in response to the acquisition of the supplementary information, the patient information being information unique to the subject.

### CROSS-REFERENCE TO RELATED APPLICATIONS

This application is based upon and claims the benefit of priority from Japanese Patent Application No. 2022-136553, filed on Aug. 30, 2022; the entire contents of which are incorporated herein by reference.

### FIELD

Embodiments described herein relate generally to a medical information system and a medical image diagnostic device.

### BACKGROUND

When a patient undergoes a test, a doctor, a clinical department, a hospital, and the like may be changed. In the related art, in order to mutually share patient information, various systems used in a hospital share patient information on the basis of standardized rules.

However, when a patient undergoes a test, information that is not included in the patient information, such as allergy information, may be revealed for the first time. The revealed information may not be reflected in patient information, for example, stored in an electronic medical record.

### BRIEF DESCRIPTION OF THE DRAWINGS

FIG. 1 is a block diagram illustrating an example of the configuration of a medical information system according to an embodiment;

FIG. 2 is a diagram illustrating an example of a data structure of supplementary information according to an embodiment;

FIG. 3 is a diagram illustrating an example of the functional configuration of a console according to an embodiment;

FIG. 4 is a diagram illustrating an example of the functional configuration of a radiology information server according to an embodiment; and

FIG. 5 is a sequence illustrating an example of processing content of the medical information system according to an embodiment.

### DETAILED DESCRIPTION

A medical information system according to an embodiment includes a first processor on a medical image diagnostic device side. The first processor acquires supplementary information, which is new information on a subject to be imaged, as structured information associated with image data acquired using the medical image diagnostic device. In response to the acquisition of the supplementary information, the first processor notifies at least one management server that manages patient information, which is information unique to the subject, of the supplementary information via a network.

The medical information system related to the present embodiment is described below with reference to the drawings. In the following embodiment, since parts denoted by the same reference numerals are assumed to perform the same operations, redundant description thereof is omitted as appropriate.

### Embodiment

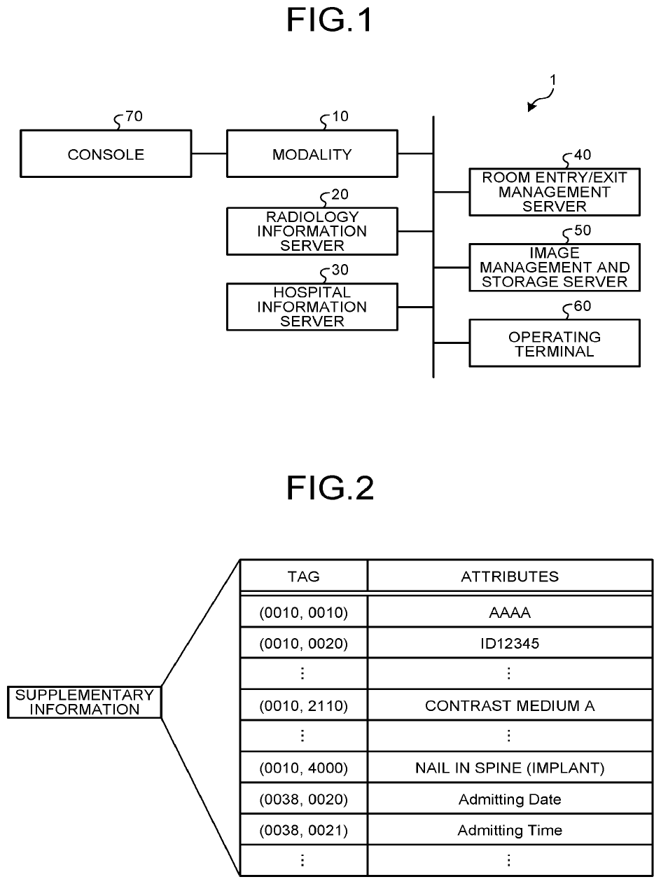

FIG. 1 is a block diagram illustrating an example of the configuration of a medical information system 1 according to the embodiment. The medical information system 1 includes a modality 10, a radiology information server 20, a hospital information server 30, a room entry/exit management server 40, an image management and storage server 50, and an operating terminal 60.

Respective systems and respective devices are communicably connected to each other via a network. The configuration illustrated in FIG. 1 is an example, and the number of respective systems and the number of respective devices may be arbitrarily changed. Devices not illustrated in FIG. 1 may also be connected to the network. That is, the medical information system 1 illustrated in FIG. 1 includes one modality 10; however, two or more modalities 10 may be provided.

The modality 10 is a medical image diagnostic device that captures images of a patient (subject) and collects medical images. The modality 10 is, for example, an X-ray computed tomography (CT) device, an X-ray diagnostic device, an ultrasonic diagnostic device, a magnetic resonance imaging device, a nuclear medicine diagnostic device, a mammography device, or the like. In the present embodiment, for the purpose of concrete explanation, the modality 10 is assumed to be an X-ray CT device.

The modality 10 as an X-ray CT device exposes a patient to be tested to X-rays according to an order from the radiology information server 20, and captures X-ray image data of the patient by using the X-rays transmitted through the patient. The modality 10 transmits the X-ray image data generated by the imaging to the radiology information server 20.

The modality 10 (X-ray CT device) according to the present embodiment includes a gantry (frame), a cradle (couch), an imaging unit, and a console 70 (operating device). The console 70 can input supplementary information for the patient.

The “supplementary information” is patient information that is newly identified and managed on the modality 10 side. The “patient information” is information unique to the patient. Examples of the supplementary information immediately prior to actual CT imaging of the patient may include information such as “allergic to contrast agent A” or “has an implant”.

Examples of the patient information may include information included in an electronic medical record such as patient ID, gender, age, medical history, doctor's findings, and medication history, medical images related to the patient acquired in the past, orders used to acquire the medical images (including test purpose, imaging site, imaging conditions, and the like), and test results for the patient. For example, the patient information is managed by the hospital information server 30 in the case of an electronic medical record, by the image management and storage server 50 in the case of image data, and by the radiology information server 20 in the case of order information.

The supplementary information can be input by the console 70 of the modality 10, for example, as digital imaging and communication in medicine (DICOM) structured data (tag information). The console 70 also performs image reconstruction processing by using projection data acquired by imaging to generate X-ray CT images.

The DICOM tag is described below with reference to FIG. 2. FIG. 2 is a diagram illustrating an example of a data structure of the supplementary information.

Specifically, the supplementary information is associated with attributes for each DICOM tag. The attributes are information indicating the content of patient information. For example, a DICOM tag (0010, 0010) is associated with a patient name. A patient name illustrated in FIG. 2 is “AAAA”. For example, a DICOM tag (0010, 0020) is associated with a patient ID (patient identifier) that can identify a patient. A patient ID illustrated in FIG. 2 is “ID12345”.

For example, a DICOM tag (0010, 2110) is associated with allergy information on patient allergies. The allergy information is information for, when a contrast agent used in the modality 10 causes side effects, specifying the contrast agent. When a subject has an allergy, appending this information as patient information is beneficial because imaging conditions need to be set in consideration of the allergy during a test of the modality 10. Allergy information illustrated in FIG. 2 is “contrast agent A”.

For example, a DICOM tag (0010, 4000) is associated with comments about the patient, and for example, indicates implant information. The implant information is information for, when a subject to be tested has an implant, specifying the implant. When the subject has an implant, appending this information as patient information is beneficial because imaging conditions need to be set in consideration of the implant during a test of the modality 10. Implant information illustrated in FIG. 2 is “nail in spine (implant)”.

For example, attributes of DICOM tags (0038, 0020) and (0038, 0021) indicate the update date and update time. The update date and update time are identification information for specifying the date and time when the patient information has been updated. The update date and update time are the date and time when a medical worker has input and updated the patient information after the completion of a test by the modality 10.

The update date and update time may also be taken as an exit time when a patient leaves a test room because a medical worker inputs patient information after the completion of a test of a subject. By ascertaining the exit time, a medical worker can ascertain the place where a patient has stopped by and manage patient's room entry/exit. An update date illustrated in FIG. 2 is “Admitting Date” and an update time is “Admitting Time”.

In the supplementary information including the allergy information, the implant information, and the exit time (update date), attributes are associated with each DICOM tag, so that patient information notified by various systems in the medical information system 1 can be acquired (interpreted) and patient information of the various systems can be updated. The supplementary information is not limited thereto.

Return now back to FIG. 1. The radiology information server 20 manages various information on a radiology department. The radiology information server 20 is an example of a management server. That is, the radiology information server 20 is a server device that manages a radiology information system (RIS). The radiology information server 20 is implemented by computer equipment such as a workstation.

The radiology information server 20 further stores patient-specific information (for example, a patient ID and the like), exposure dose record information, and the like on a patient to be tested in its own storage circuitry or the like. The exposure dose record information is information on recording of the exposure dose of the patient as a result of the acquisition of X-ray image data by the modality 10.

The radiology information server 20 receives orders from the operating terminal 60, and manages tests in the radiology department according to the orders. The radiology information server 20 transmits the received orders to the modality 10.

The hospital information server 30 manages various patient information such as patients and an electronic medical record for each patient. The hospital information server 30 is an example of a management server. The hospital information server 30 is, for example, a hospital information system (HIS). The hospital information server 30 is implemented by computer equipment such as a workstation. The hospital information server 30 stores electronic medical records and test orders in its own storage circuitry or the like. The electronic medical records are data with patient medical records. The test order is data requesting the imaging of a patient by the modality 10.

The room entry/exit management server 40 manages various information on patient's room entry/exit. The room entry/exit management server 40 is an example of a management server. The room entry/exit management server 40 is implemented by computer equipment such as a workstation. The room entry/exit management server 40 stores, for example, information on patient's room entry/exit, and information on the date and time when a patient was tested by the modality 10, in its own storage circuitry or the like.

The image management and storage server 50 is a server device that stores image data such as X-ray image data generated by the modality 10. The image management and storage server 50 is an example of a management server. The image management and storage server 50 is, for example, a picture archiving and communication system (PACS). The image management and storage server 50 is implemented by computer equipment such as a workstation.

The operating terminal 60 is a terminal operated by a medical worker such as a doctor. The operating terminal 60 is an example of a medical processing device. The operating terminal 60 is implemented by computer equipment such as a personal computer or a tablet terminal. The operating terminal 60 is, for example, a terminal that inputs test orders and notifies the radiology information server 20 of the test orders.

The configuration of the console 70 according to the present embodiment is described below.

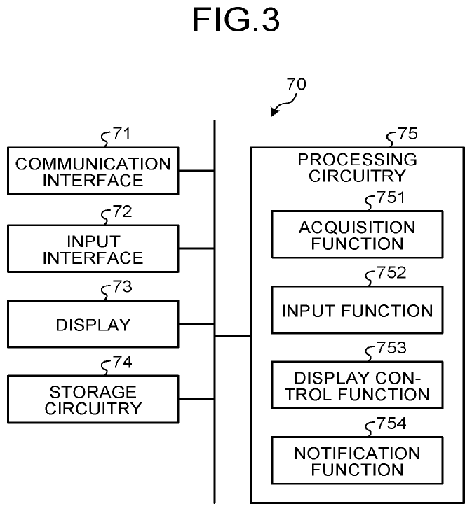

FIG. 3 is a block diagram illustrating an example of the configuration of the console 70 according to the present embodiment. The console 70 includes a communication interface 71, an input interface 72, a display 73, storage circuitry 74, and processing circuitry 75. The configuration of the console 70 is not limited thereto.

The communication interface 71 is connected to the processing circuitry 75, and controls transmission and communication of various data transmitted/received between each device connected via the network. For example, the communication interface 71 is implemented by a network card, a network adapter, a network interface controller (NIC), or the like.

The input interface 72 is connected to the processing circuitry 75, converts input operations received from an operator (medical worker) into electrical signals, and outputs the electrical signals to the processing circuitry 75. Specifically, the input interface 72 converts the input operations received from the operator into the electrical signals, and outputs the electrical signals to the processing circuitry 75.

The input interface 72 is implemented by, for example, a trackball, a switch button, a mouse, a keyboard, a touch pad for performing an input operation by touching an operation surface, a touch screen with integrated display screen and touch pad, non-contact input circuitry using an optical sensor, voice input circuitry, or the like.

In the present specification, the input interface 72 is not limited to only those with physical operation parts such as a mouse and a keyboard. An example of the input interface 72 also includes electrical signal processing circuitry that receives an electrical signal corresponding to an input operation from an external input device provided separately from the device and outputs the electrical signal to control circuitry.

The display 73 is connected to the processing circuitry 75 and displays various information and various image data output from the processing circuitry 75. For example, the display 73 is implemented by a liquid crystal display, a cathode ray tube (CRT) display, an organic electroluminescence display (OLED) display, a plasma display, a touch screen, or the like.

The storage circuitry 74 is connected to the processing circuitry 75 and stores various data. The storage circuitry 74 is an example of a storage unit. The storage circuitry 74 stores patient information of a subject. The storage circuitry 74 further stores various computer programs to be read and executed by the processing circuitry 75 to perform various functions. For example, the storage circuitry 74 is implemented by a semiconductor memory element such as a random access memory (RAM) and a flash memory, a hard disk, an optical disk, or the like.

The processing circuitry 75 controls the entire operation of the console 70. The processing circuitry 75 includes, for example, an acquisition function 751, an input function 752, a display control function 753, and a notification function 754. In the embodiment, respective processing functions performed by the acquisition function 751, the input function 752, the display control function 753, and the notification function 754 are stored in the storage circuitry 74 in the form of computer programs executable by a computer.

The processing circuitry 75 is a processor that reads the computer programs from the storage circuitry 74 and executes the read computer programs, thereby implementing the functions corresponding to the executed computer programs. In other words, the processing circuitry 75 in the state of reading the computer programs has the functions illustrated in the processing circuitry 75 in FIG. 3.

In FIG. 3, the acquisition function 751, the input function 752, the display control function 753, and the notification function 754 are described as being implemented by a single piece of processor; however, a plurality of independent processors may be combined to form the processing circuitry 75 and the functions may be implemented by each processor executing the computer program.

In FIG. 3, single piece of storage circuitry such as the storage circuitry 74 is described as storing the computer programs corresponding to the respective processing functions; however, a plurality of pieces of storage circuitry may be distributedly disposed and the processing circuitry 75 may be configured to read each computer program from each storage circuitry and execute the read computer program.

The term “processor” used in the above description, for example, means circuitry such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)).

The processor implements functions by reading and executing the computer programs stored in the storage circuitry 74. Instead of storing the computer programs in the storage circuitry 74, the computer programs may be directly incorporated in the circuitry of the processor. In this case, the processor implements the functions by reading and executing the computer programs incorporated in the circuitry.

The acquisition function 751 acquires various information from the hospital information server 30. The acquisition function 751 is an example of an acquisition unit. The acquisition function 751 acquires electronic medical records from the hospital information server 30.

The acquisition function 751 further acquires supplementary information, which is new information on a subject to be imaged, as structured information associated with image data acquired using the modality 10. The acquisition function 751 further acquires the supplementary information as DICOM tags for the image data.

The input function 752 inputs the supplementary information. The input function 752 inputs, as information of the image data acquired using the modality 10, the supplementary information, which is new information on the subject to be imaged that is not included in the patient information included in the electronic medical records acquired by the acquisition function 751, before the imaging by the modality 10 is performed.

The supplementary information is, for example, allergy information and implant information. The input function 752 inputs the supplementary information, and inputs the date of update of the patient information. The update date may be acquired by the input function 752 from, for example, a real-time clock (RTC) of the console 70, and may be assigned to the patient information. The functions of the input function 752 are not limited thereto.

The display control function 753 allows the display 73 to display various screens. The display control function 753 is an example of a display control unit. The display control function 753, for example, displays the electronic medical records acquired by the acquisition function 751. The display control function 753, for example, displays the patient information input by the input function 752. The display control function 753 may display other information as well the as the patient information.

The notification function 754 determines whether the patient information includes the supplementary information. The notification function 754 is an example of a notification unit. The notification function 754 determines whether the patient information input by the input function 752 includes new supplementary information. When the patient information input by the input function 752 includes the new supplementary information, the notification function 754 notifies the radiology information server 20 and the image management and storage server 50 of the supplementary information via the network.

When a tag (for example, a DICOM tag) for specifying the supplementary information is attached, the notification function 754 notifies the radiology information server 20 and the image management and storage server 50 of the supplementary information. The notification function 754 further notifies the radiology information server 20 and the image management and storage server 50 of an update notification for updating the patient information. The notification function 754 may notify other management servers of the patient information as well as the radiology information server 20 and the image management and storage server 50.

The notification function 754 can notify the radiology information server 20 of the newly input supplementary information, thereby promoting an update of the patient information recorded on the radiology information server 20 and the Image management and storage server 50. The radiology information server 20 and the image management and storage server 50 can be notified of the patient information including the supplementary information, thereby keeping the patient information up-to-date. Since the radiology information server 20 and the image management and storage server 50 can keep the patient information up-to-date, imaging conditions can be set in advance on the basis of the content included in the supplementary information at the time of a subsequent test.

The configuration of the radiology information server 20 according to the present embodiment is described below.

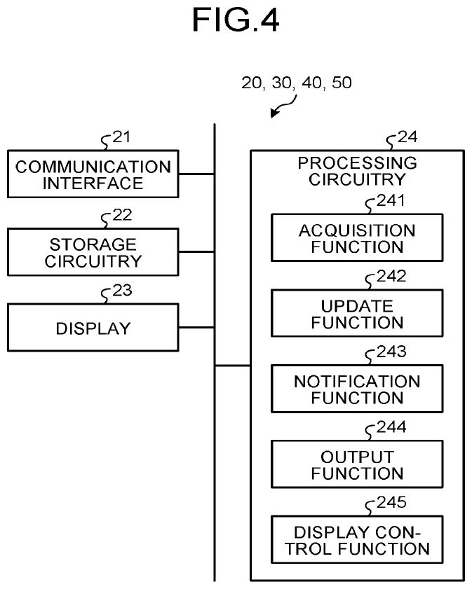

FIG. 4 is a block diagram illustrating an example of the configuration of the radiology information server 20 according to the present embodiment. The radiology information server 20 includes a communication interface 21, storage circuitry 22, a display 23, and processing circuitry 24. The configuration of the radiology information server 20 is not limited thereto.

The configurations of the hospital information server 30, the room entry/exit management server 40, and the image management and storage server 50 are the same as the configuration of the radiology information server 20. Therefore, for the configurations of the hospital information server 30, the room entry/exit management server 40, and the image management and storage server 50, description of the configuration of the radiology information server 20 is referred to.

The communication interface 21 is connected to the processing circuitry 24, and controls transmission and communication of various data transmitted/received between each device connected via the network. For example, the communication interface 21 is implemented by a network card, a network adapter, a network interface controller (NIC), or the like.

The storage circuitry 22 is connected to the processing circuitry 24 and stores various data. The storage circuitry 22 is an example of a storage unit. The storage circuitry 22 stores patient information of a subject. The storage circuitry 22 stores various computer programs to be read and executed by the processing circuitry 24 to perform various functions. For example, the storage circuitry 22 is implemented by a semiconductor memory element such as a random access memory (RAM) and a flash memory, a hard disk, an optical disk, or the like.

A display 23 is connected to the processing circuitry 24, and displays various information and various image data output from the processing circuitry 24. For example, the display 23 is implemented by a liquid crystal display, a cathode ray tube (CRT) display, an organic electroluminescence display (OLED) display, a plasma display, a touch panel, or the like.

The processing circuitry 24 controls the entire operation of the radiology information server 20. The processing circuitry 24 includes, for example, an acquisition function 241, an update function 242, and a notification function 243. In the embodiment, respective processing functions performed by the acquisition function 241, the update function 242, and the notification function 243 are stored in the storage circuitry 22 in the form of computer programs executable by a computer.

The processing circuitry 24 is a processor that reads the computer programs from the storage circuitry 22 and executes the read computer programs, thereby implementing the functions corresponding to the executed computer programs. In other words, the processing circuitry 24 in the state of reading the computer programs has the functions illustrated in the processing circuitry 24 in FIG. 4.

In FIG. 4, the acquisition function 241, the update function 242, and the notification function 243 are described as being implemented by a single piece of processor; however, a plurality of independent processors may be combined to form the processing circuitry 24 and the functions may be implemented by each processor executing the computer program.

In FIG. 4, single piece of storage circuitry such as the storage circuitry 22 is being described as storing the computer programs corresponding to the respective processing functions; however, a plurality of pieces of storage circuitry may be distributedly disposed and the processing circuitry 24 may be configured to read each computer program from each storage circuitry and execute the read computer program.

The term “processor” used in the above description, for example, means circuitry such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)).

The processor implements functions by reading and executing the computer programs stored in the storage circuitry 22. Instead of storing the computer programs in the storage circuitry 22, the computer programs may be directly incorporated in the circuitry of the processor. In this case, the processor implements the functions by reading and executing the computer programs incorporated in the circuitry.

The processing circuitry 24 of the hospital information server 30 controls the operation of the entire hospital information server 30. The processing circuitry 24 of the room entry/exit management server 40 controls the operation of the entire room entry/exit management server 40. The processing circuitry 24 of the image management and storage server 50 controls the operation of the entire image management and storage server 50.

The acquisition function 241 acquires the supplementary information from the console 70. The acquisition function 241 is an example of an acquisition unit. The acquisition function 241 acquires the supplementary information from the console 70 via the network. The acquisition function 241 stores the acquired supplementary information in the storage circuitry 22.

The acquisition function 241 of the hospital information server 30 acquires the supplementary information from the radiology information server 20 via the network. The acquisition function 241 of the room entry/exit management server 40 acquires the supplementary information from the hospital information server 30 via the network. The acquisition function 241 of the image management and storage server 50 acquires the supplementary information from the console 70 via the network.

The update function 242 updates patient information. The update function 242 is an example of an update unit. On the basis of the supplementary information acquired by the acquisition function 241, the update function 242 updates the patient information stored in the storage circuitry 22. Thus, the update function 242 can keep the patient information up-to-date.

The notification function 243 determines whether the patient information includes the supplementary information. The notification function 243 is an example of a notification unit. The notification function 243 determines whether the patient information acquired by the acquisition function 241 includes new supplementary information. When the patient information acquired by the acquisition function 241 includes the new supplementary information, the notification function 243 notifies the hospital information server 30 of the supplementary information.

When a tag (for example, a DICOM tag) for specifying the supplementary information is attached, the notification function 243 notifies the hospital information server 30 of the supplementary information. The notification function 243 further notifies the hospital information server 30 of an update notification for updating the patient information. The notification function 243 may notify other management servers of the patient information as well as the hospital information server 30.

The notification function 243 can notify the hospital information server 30 of the newly input supplementary information, thereby promoting an update of the patient information recorded on the hospital information server 30. The hospital information server 30 can be notified of the patient information including the supplementary information, thereby keeping the patient information up-to-date.

When the patient information acquired by the acquisition function 241 of the hospital information server 30 includes new supplementary information, the notification function 243 of the hospital information server 30 notifies the room entry/exit management server 40 of the supplementary information. When a tag (for example, a DICOM tag) for specifying the supplementary information is attached, the notification function 243 of the hospital information server 30 notifies the room entry/exit management server 40 of the supplementary information. The notification function 243 of the hospital information server 30 further notifies the room entry/exit management server 40 of an update notification for updating the patient information.

The notification function 243 of the hospital information server 30 can notify the room entry/exit management server 40 of the newly input supplementary information, thereby promoting an update of the patient information recorded on the room entry/exit management server 40. The room entry/exit management server 40 can be notified of the patient information including the supplementary information, thereby keeping the patient information up-to-date.

When the patient information acquired by the acquisition function 241 of the image management and storage server 50 includes new supplementary information, the notification function 243 of the image management and storage server 50 notifies the hospital information server 30 of the supplementary information. When a tag (for example, a DICOM tag) for specifying the supplementary information is attached, the notification function 243 of the image management and storage server 50 notifies the hospital information server 30 of the supplementary information. The notification function 243 of the image management and storage server 50 further notifies the hospital information server 30 of an update notification for updating the patient information.

The notification function 243 of the image management and storage server 50 can notify the hospital information server 30 of the newly input supplementary information, thereby promoting an update of the patient information recorded on the hospital information server 30. The hospital information server 30 can be notified of the patient information including the supplementary information, thereby keeping the patient information up-to-date.

When the patient information is updated by the update function 242, a output function 244 outputs information notifying the update. The output function 244 is an example of an output unit. When the patient information is updated by the update function 242, the output function 244 outputs a pop-up to the display 23.

A display control function 245 allows the display 23 to display various screens. The display control function 245 is an example of a display control unit. The display control function 245, for example, displays the supplementary information acquired by the acquisition function 241. The display control function 245, for example, displays the pop-up output by the output function 244.

A patient information update process performed by the medical information system 1 according to the embodiment is described below. In the patient information update process performed by the medical information system 1, upon the execution of imaging by the modality 10 side according to received order information, when new information (supplementary information) on a patient is generated, the modality 10 side notifies each server that manages patient information of the patient of the supplementary information, and the patient information is automatically updated.

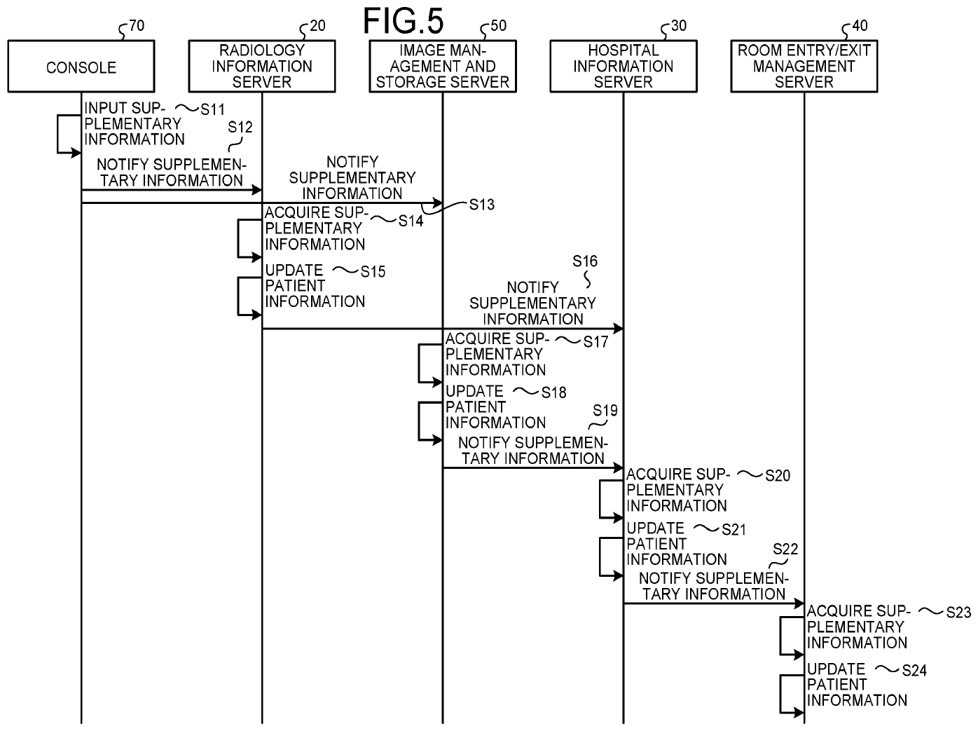

FIG. 5 is a sequence illustrating an example of the patient information update process performed by the medical information system 1. For the purpose of concrete explanation, the following assumes that the modality 10 side generates supplementary information that a patient is “allergic to contrast agent A” before a test by the modality 10, and patient information is updated using the supplementary information after the completion of the test by the modality 10.

The input function 752 of the console 70 cooperates with the input interface 72 of the console 70 to input supplementary information, which is new information on a subject to be imaged, as tag information of a DICOM tag of image data acquired using the modality 10 (step S11). Subsequently, the acquisition function 751 of the console 70 acquires the supplementary information, which is the new information on the subject to be imaged, as structured information (tag information of the DICOM tag) associated with the image data acquired using the modality 10.

Subsequently, the notification function 754 of the console 70 notifies the radiology information server 20 of the supplementary information via the network, for example, together with a patient ID for specifying the patient (step S12). Subsequently, the notification function 754 of the console 70 notifies the image management and storage server 50 of the supplementary information via the network, for example, together with a patient ID for specifying the patient (step S13).

The acquisition function 241 of the radiology information server 20 acquires the supplementary information notified by the notification function 754 of the console 70, and stores the supplementary information in the storage circuitry 22 (step S14). Subsequently, the update function 242 of the radiology information server 20 updates patient information (order information for the patient) stored in the storage circuitry 22, on the basis of the supplementary information acquired by the acquisition function 241 (step S15). Subsequently, a notification function 231 of the radiology information server 20 notifies the hospital information server 30 of the supplementary information via the network together with the patient ID of the patient (step S16).

The acquisition function 241 of the image management and storage server 50 acquires the supplementary information notified by the notification function 754 of the console 70, and stores the supplementary information in the storage circuitry 22 (step S17). Subsequently, on the basis of the supplementary information acquired by the acquisition function 241, the update function 242 of the image management and storage server 50 updates the patient information (order information for the patient) stored in the storage circuitry 22 (step S18). Subsequently, a notification function 231 of the image management and storage server 50 notifies the hospital information server 30 of the supplementary information via the network together with the patient ID of the patient (step S19).

The acquisition function 241 of the hospital information server 30 acquires the supplementary information notified by the notification function 231 of at least one of the radiology information server 20 and the image management and storage server 50, and stores the supplementary information in the storage circuitry 22 (step S20). Subsequently, on the basis of the supplementary information acquired by the acquisition function 241, the update function 242 of the hospital information server 30 updates the patient information (electronic medical record) stored in the storage circuitry 22 (step S21). Subsequently, a notification function 231 of the hospital information server 30 notifies the room entry/exit management server 40 of the supplementary information via the network together with the patient ID of the patient (step S22).

The acquisition function 241 of the room entry/exit management server 40 acquires the supplementary information notified by the notification function 231 of the hospital information server 30, and stores the supplementary information in the storage circuitry 22 (step S23). Subsequently, on the basis of the supplementary information (exit time) acquired by the acquisition function 241, the update function 242 of the room entry/exit management server 40 updates the patient information stored in the storage circuitry 22 (step S24).

As described above, in the medical information system 1 according to the embodiment, the modality 10 side inputs supplementary information, which is new information on a subject to be imaged, as information of image data acquired using the modality 10, and notifies at least one management server (radiology information server 20) that manages patient information, which is information unique to the subject, of the supplementary information via the network.

According to at least one embodiment described above, for example, supplementary information as patient information newly revealed at the time of a test is input as, for example, DICOM tag information, so that patient information managed by each management server used in a hospital can be automatically updated to new patient information including the supplementary information. This allows the management server to automatically keep patient information stored in the storage circuitry 22 up-to-date without leaking the supplementary information. Furthermore, the management server automatically updates the patient information, which reduces the workload of a medical worker.

Moreover, a medical worker can perform subsequent processing by using patient information updated to reflect the supplementary information, thereby suppressing leakage of information such as allergy information on a contrast agent and performing a safe test. Furthermore, a medical worker can set imaging conditions based on the supplementary information.

In the above embodiment, the console 70 is described as including the acquisition function 751, the input function 752, the display control function 753, and the notification function 754. However, the modality 10 may also include all or part of acquisition function 751, the input function 752, the display control function 753, and the notification function 754.

### First Modification

In the above embodiment, a case in which supplementary information is input as DICOM tag information by using the console 70 of the modality 10 and registered in medical information such as an electronic medical record is used as an example. On the other hand, the supplementary information may be input as DICOM tag information by using a tablet, a personal computer, or the like used by the modality 10 side.

### Second Modification

In the above embodiment, a case in which supplementary information is input as DICOM tag information by using the console 70 of the modality 10 and registered in medical information such as an electronic medical record is used as an example. On the other hand, when there are differences in medical information stored in various management servers (for example, the medical information is updated), the various management servers may notify a user using the management servers of whether to update the medical information, in order to keep the medical information up-to-date.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information system comprising:
a first processor on a side of a medical image diagnostic device, wherein
the first processor
acquires supplementary information as structured information associated with image data acquired using the medical image diagnostic device, the supplementary information being new information on a subject to be imaged, and
notifies at least one management server that manages patient information of the supplementary information via a network in response to the acquisition of the supplementary information, the patient information being information unique to the subject.

2. The medical information system according to claim 1, wherein
the at least one management server includes a second processor, and
the second processor
acquires the supplementary information via the network,
stores patient information of the subject, and
updates the stored patient information on the basis of the acquired supplementary information.

3. The medical information system according to claim 1, wherein the first processor acquires the supplementary information as a DICOM tag of the image data.

4. The medical information system according to claim 2, wherein, when the patient information is updated, the second processor outputs information notifying the update.

5. The medical information system according to claim 1, wherein the supplementary information includes at least one of allergy information and implant information.

6. The medical information system according to claim 5, wherein the supplementary information includes an exit time when the subject leaves a test room.

7. The medical information system according to claim 1, wherein the management server includes a radiology management server and an image management and storage server.

8. A medical image diagnostic device comprising:
an imaging unit; and
a processor, wherein
the processor
acquires supplementary information as structured information associated with image data acquired using the imaging unit, the supplementary information being new information on a subject to be imaged, and
notifies at least one management server that manages patient information of the supplementary information via a network in response to input of the supplementary information, the patient information being information unique to the subject.
